(19) United States
(12) Patent Application Publication
(10) Pub. No.: US 2010/0099921 A1
(43) Pub. Date: Apr. 22, 2010

(54) Method for Producing Alkylene Glycol Diethers

(76) Inventors: Gabriele Oberendfellner, Sulzbach (DE); Alexander Snell, Sulzbach (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 11/989,991

(22) PCT Filed: Jul. 8, 2006
(86) PCT No.: PCT/EP2006/006695

§ 371 Date: Feb. 1, 2008

(30) Foreign Application Priority Data
Aug. 10, 2005 (DE) 10 2005 037 760.2

(51) Int. Cl. C07C 41/02

(52) U.S. Cl. 568/613

(57) ABSTRACT

The invention relates to a method for producing alkylene glycol diethers by reacting a linear or cyclic ether with an alkylene oxide in the presence of a Lewis acid. The method is characterized in that the Lewis acid is a mixture of 1 part by weight of HBF₄ and/or BF₃ and 0.1 to 10 parts by weight of H₂SO₄, HNO₃ and/or H₃PO₄. The new catalyst system allows to reduce undesired by-products such as e.g. dioxan or dimethyltriethylene glycol, and to increase the quantity of valuable substances such as dimethyl glycol and dimethyl diglycol.

The present invention relates to a process for preparing catenated alkylene glycol diethers by means of a novel catalyst system.

Alkylene glycol diethers have been used for some time as polar inert solvents. For their preparation, indirect processes, for example the Williamson ether synthesis (K. Weissermel, H. J. Arpe, “Industrielle Organische Chemie” [Industrial Organic Chemistry], 1998, page 179) or the hydrogenation of diglycol ether formal (DE-A-24 34 057) are employed industrially or described, and also direct processes, for example the insertion of alkylene oxide into a catenated ether in the presence of Lewis acids such as BF₃ (U.S. Pat. No. 4,146,736, DE-A-2 741 676 and DE-A-2 640 505 in conjunction with DE-A-3 128 962) or SnCl₄ (DE-A-3 025 434).

The technical advantage of the direct processes lies not only in a simplification of the preparation process, but also in that no by-products such as large amounts of sodium chloride or sodium sulfate are formed as in the Williamson synthesis, or glycol ethers as in the formal hydrogenation. They are therefore economically significantly less expensive processes.

One disadvantage of the direct processes is, according to DE-A-3 025 434, that a large amount of cyclic alkylene oxide dimers (for example 1,4-dioxane) is unavoidably formed. These cyclic dimers form through cyclization of 2 molecules of alkylene oxide. DE-A-3 025 434 therefore describes a process in which tin(IV) chloride or boron trifluoride are used together with compounds having active hydrogen as catalyst systems. The compounds having active hydrogen listed are, as well as water, also various alcohols and various organic acids. The amount of dioxane obtained in this process is between 12.9 and 24.4%. However, the disadvantage of this process is the very wide molar mass distribution of the different polyglycol dimethyl ethers, which have to be separated from one another in a complicated manner.

DE-A-2 741 676 describes the use of metal halides, for example boron trifluoride, in conjunction with boric acids, preferably orthoboric acid H₃BO₃, as catalysts. In this process, the dioxane content can be lowered to 3.8%, and the molar mass distribution is less wide than in the process according to DE-A-3 025 434. In both processes, however, between 10 and 15% dimethyltriethylene glycol is formed, which can be sold only with difficulty owing to its high boiling point of 275° C.

To improve the yield and the selectivity of preparation of polyalkylene glycol dialkyl ethers from dialkyl ethers, new catalyst compositions are therefore required.

It has now been found that, surprisingly, mixtures of particular catalysts known per se significantly improve both the yield and the selectivity of the insertion reaction. With the novel catalyst system, a lower level of undesired by-products is formed, for example dioxane or dimethyltriethylene glycol, and a higher proportion of the substances of value dimethylglycol and dimethyldiglycol.

The present invention therefore provides a process for preparing alkylene glycol diethers by reacting a linear or cyclic ether with an alkylene oxide in the presence of a Lewis acid, wherein the Lewis acid is a mixture of 1 part by weight of HBF₄ and/or BF₃ and from 0.1 to 10 parts by weight of H₂SO₄, HNO₃ and/or H₃PO₄.

In the process according to the invention, the linear or cyclic ethers, the alkylene oxide and the required Lewis acid are metered into the reactor in liquid form (if required under pressure). The reaction is performed at a pressure of from 0 to 30 bar (above standard pressure), preferably at a pressure of from 8 to 20 bar, and at a temperature of from 0° C. to 200° C., preferably from 20° C. to 100° C. After the conversion of the reactants, the reaction mixture comprising the product formed is brought to standard pressure by means of a decompression vessel and then worked up.

The ethers which may be used as starting materials for the process according to the invention include various ethers with lower alkyl groups, and especially those of the formula 1

R¹—O—R² (1)

in which R¹ is a C₁ to C₁₂-alkyl group, R² is a C₁ to C₁₂-alkyl group or a phenyl or benzyl group, or in which R¹ and R², with inclusion of the oxygen atom, form a ring having 5, 6 or 7 atoms.

Preferably R¹ and R² are each independently C₁ to C₄-alkyl, especially methyl or ethyl.

When R¹ and R² form a ring, it corresponds to the formula

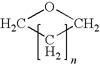

in which n is 2, 3 or 4. A preferred cyclic compound is tetrahydrofuran.

Various alkylene oxides can be used in accordance with the invention. Preference is given to the compounds of the formula 2

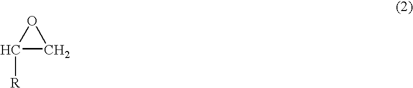

in which R is hydrogen, halogen, an alkyl group having from 1 to 10 carbon atoms, a phenyl group or a benzyl group.

Examples of suitable alkylene oxides are ethylene oxide, propylene oxide, butylene oxide, epichlorohydrin, styrene oxide and the mixture of these compounds. Particular preference is given to ethylene oxide and propylene oxide.

The compounds obtained by the process according to the invention correspond to the formula

R¹—O—[—(CH₂)ₓ—O]_{y}—R²

in which, each independently,

R¹ is C₁ to C₁₂-alkyl

R² is C₁ to C₁₂-alkyl, or a phenyl group or benzyl group,

x is an integer from 1 to 6

y is an integer from 1 to 20.

Preferably, R¹ and R² are each a methyl or ethyl group, especially a methyl group.

The novel catalyst comprises firstly HBF₄ and/or BF₃, and secondly H₂SO₄, HNO₃ and/or H₃PO₄, in a weight ratio of 1: (0.1-10), preferably 1: (0.3-5), especially 1: (0.5-3).

When firstly HBF₄ and BF₃, and/or secondly at least 2 acids selected from H₂SO₄, HNO₃ and H₃PO₄, are used in a mixture as the catalyst, the above-specified weight ratios apply to these mixtures.

Particularly preferred acids are H₂SO₄ and H₃PO₄.

It is possible to employ solvents in the process according to the invention when they give rise to advantages in the preparation of catalysts, for example to an increase in the solubility, and/or to an increase/reduction in the viscosity and/or to the removal of heat of reaction. Examples thereof are inert solvents such as dichloromethane, nitromethane, benzene, toluene, acetone, ethyl acetate, or dioxane or active solvents such as methanol, ethanol, propanol, butanol, methylglycol, methyldiglycol, methyltriglycol, or the target substances themselves, such as mono-, di-, tri-, tetra- or polyalkylene glycol dimethyl ether.

In the process according to the invention, it is possible to prepare alkylene glycol diethers in good yield in a continuous or batchwise process.

### EXAMPLES

Comparative Example 1

### HBF₄ Catalysis

A nitrogen-purged 1 l steel autoclave is initially charged with 200 mg (2.28 mmol) of fluoroboric acid and 157 g (3.41 mol) of dimethyl ether. At 55° C. and 15 bar, 15.0 g (0.34 mmol) of ethylene oxide are added rapidly. After the pressure has fallen to 13 bar, stirring is continued at 55° C. for another 50 min. After the excess dimethyl ether has been given out, what remains is a liquid residue of 24.4 g with the following composition:

53.0% dimethylethylene glycol

7.6% 1,4-d ioxane

3.3% methylglycol

15.3% dimethyldiethylene glycol

4.6% methyldiglycol

5.0% dimethyltriethylene glycol

2.8% methyltriglycol

0.2% methyltetraglycol

8.2% unknown

### Comparative Example 2

### BF₃ Catalysis

A nitrogen-purged 1 l steel autoclave is initially charged with 297 mg (2.61 mmol) of boron trifluoride dimethyl etherate and 157 g (3.41 mol) of dimethyl ether. At 55° C. and 15 bar, 15.0 g (0.34 mol) of ethylene oxide are added rapidly. After the pressure has fallen to 13 bar, stirring is continued at 55° C. for another 50 min. After the excess dimethyl ether has been given out, what remains is a liquid residue of 5.40 g with the following composition:

55.7% dimethylethylene glycol

9.4% 1,4-d ioxane

1.0% methylglycol

15.4% dimethyldiethylene glycol

3.3% methyldiglycol

5.7% dimethyltriethylene glycol

1.8% methyltriglycol

2.5% dimethyltetraglycol

0.2% methyltetraglycol

0.9% dimethylpentaglycol

4.1% unknown

### Comparative Example 3

### H₂SO₄ Catalysis

A nitrogen-purged 1 l steel autoclave is initially charged with 461 mg (4.56 mmol) of sulfuric acid and 157 g (3.41 mol) of dimethyl ether. At 55° C. and 15 bar, 15.0 g (0.34 mol) of ethylene oxide are added rapidly. After the pressure has fallen to 13 bar, stirring is continued at 55° C. for another 50 min and then the excess dimethyl ether is driven out. 0.7 g of a liquid residue can be isolated, which has the following composition:

0.0% dimethylethylene glycol

5.1% 1,4-dioxane

17.2% methylglycol

1.2% dimethyldiethylene glycol

8.6% methyldiglycol

3.7% dimethyltriethylene glycol

0.0% methyltriglycol

35.1% dimethyltetraglycol

29.1% unknown

### Example 4

### HBF₄/H₂SO₄ Catalysis

Analogously to comparative example 1, a 1 l steel autoclave is initially charged with 200 mg (2.28 mmol) of fluoroboric acid, 231 mg (2.28 mmol) of sulfuric acid and 157 g (3.41 mol) of dimethyl ether, and 15.0 g (0.34 mol) of ethylene oxide are added at 55° C. and 15 bar. After 50 min of continued reaction time and driving out the dimethyl ether, 30.7 g of residue are isolated. The residue has the following composition:

59.9% dimethylethylene glycol

2.1% 1,4-dioxane

6.8% methylglycol

17.8% dimethyldiethylene glycol

3.0% methyldiglycol

4.3% dimethyltriethylene glycol

0.7% methyltriglycol

1.2% dimethyltetraglycol

0.3% dimethylpentaglycol

3.9% unknown

### Example 5

### HBF₄/H₃PO₄ Catalysis

Analogously to comparative example 1, a 1 l steel autoclave is initially charged with 200 mg (2.28 mmol) of fluoroboric acid, 223 mg (2.28 mmol) of phosphoric acid and 157 g (3.41 mol) of dimethyl ether, and 15.0 g (0.34 mol) of ethylene oxide are added at 55° C. and 15 bar. After 50 min of continued reaction time and driving out the dimethyl ether, 23.8 g of residue are isolated. The residue has the following composition:

60.1% dimethylethylene glycol

1.7% 1,4-dioxane

5.0% methylglycol

16.9% dimethyldiethylene glycol

4.6% methyldiglycol

3.8% dimethyltriethylene glycol

1.6% methyltriglycol

0.9% dimethyltetraglycol

1.4% methyltetraglycol

0.1% dimethylpentaglycol

3.9% unknown

### Example 6

### HBF₄/HNO₃ Catalysis

Analogously to comparative example 1, a 1 l steel autoclave is initially charged with 200 mg (2.28 mmol) of fluoroboric acid, 288 mg (2.96 mol) of nitric acid and 157 g (3.41 mol) of dimethyl ether, and 15.0 g (0.34 mol) of ethylene oxide are added at 55° C. and 15 bar. After 50 min of continued reaction time and driving out the dimethyl ether, 29.7 g of residue are isolated. The residue has the following composition:

59.8% dimethylethylene glycol

1.8% 1,4-dioxane

3.0% methylglycol

16.3% dimethyldiethylene glycol

4.3% methyldiglycol

3.8% dimethyltriethylene glycol

1.9% methyltriglycol

3.2% dimethyltetraglycol

1.1% methyltetraglycol

0.3% dimethylpentaglycol

4.5% unknown

### Example 7

### BF₃/H₂SO₄ Catalysis

Analogously to comparative example 1, a 1 l steel autoclave is initially charged with 200 mg (2.28 mol) of boron trifluoride dimethyl etherate, 231 mg (2.28 mol) of sulfuric acid and 157 g (3.41 mol) of dimethyl ether, and 15.0 g (0.34 mol) of ethylene oxide are added at 55° C. and 15 bar. After 50 min of continued reaction time and driving out the dimethyl ether, 12.2 g of residue are isolated. The residue has the following composition:

62.4% dimethylethylene glycol

3.9% 1,4-dioxane

3.2% methylglycol

15.2% dimethyldiethylene glycol

3.8% methyldiglycol

4.3% dimethyltriethylene glycol

0.9% methyltriglycol

1.3% dimethyltetraglycol

0.4% dimethylpentaglycol

4.6% unknown

### Example 8

### BF₃/H₃PO₄ Catalysis

Analogously to comparative example 1, a 1 l steel autoclave is initially charged with 260 mg (2.28 mmol) of boron trifluoride dimethyl etherate, 447 mg (4.56 mmol) of phosphoric acid and 157 g (3.41 mol) of dimethyl ether, and 15.0 g (0.34 mol) of ethylene oxide are added at 55° C. and 15 bar. After 50 min of continued reaction time and driving out the dimethyl ether, 30.8 g of residue are isolated. The residue has the following composition:

60.2% dimethylethylene glycol

5.3% 1,4-dioxane

4.5% methylglycol

17.3% dimethyldiethylene glycol

2.2% methyldiglycol

3.1% dimethyltriethylene glycol

2.0% dimethyltetraglycol

0.9% dimethylpentaglycol

4.5% unknown

### Example 9

### BF₃/HNO₃ Catalysis

Analogously to comparative example 1, a 1 l steel autoclave is initially charged with 260 mg (2.28 mmol) of boron trifluoride dimethyl etherate, 144 mg (1.48 mol) of nitric acid and 157 g (3.41 mol) of dimethyl ether, and 15.0 g (0.34 mol) of ethylene oxide are added at 55° C. and 15 bar. After 50 min of continued reaction time and driving out the dimethyl ether, 8.80 g of residue are isolated. The residue has the following composition:

63.1% dimethylethylene glycol

3.8% 1,4-dioxane

3.0% methylglycol

13.3% dimethyldiethylene glycol

6.0% methyldiglycol

3.6% dimethyltriethylene glycol

1.6% methyltriglycol

1.0% dimethyltetraglycol

0.3% dimethylpentaglycol

4.3% unknown

1. A process for preparing alkylene glycol diethers by reacting a linear ether of the formula
R¹—O—R²
in which R¹ is a C₁ to C₁₂-alkyl group, R² is a C₁ to C₁₂-alkyl group or a phenyl or benzyl group, with an alkylene oxide of the formula 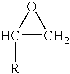
in which R is H, halogen, C₁-C₁₀-alkyl, phenyl or benzyl, in the presence of a Lewis acid, wherein the Lewis acid is a mixture of 1 part by weight of a boron compound selected from the group consisting of HBF₄, BF₃ and mixtures thereof and from 0.1 to 10 parts by weight of a mineral acid selected from the group consisting of H₂SO₄, HNO₃, H₃PO₄, and mixtures thereof.

2. The process as claimed in claim 1, in which the ratio of the boron compound to the mineral acid is 1 :(0.3-5).

3. The process of claim 1, in which the process is conducted in a solvent selected from the group consisting of dichloromethane, nitromethane, benzene, toluene, acetone, ethyl acetate, dioxane, methanol, ethanol, propanol, butanol, methylglycol, methyldiglycol, methyltriglycol, mono-glycol dimethyl ether, and polyalkylene glycol dimethyl ether.

4. The process of claim 1 in which the mineral is selected from the group consisting of H₂SO₄, H₃PO₄, and mixtures thereof.
